(19) United States
(12) Patent Application Publication
(10) Pub. No.: US 2014/0170224 A1
(43) Pub. Date: Jun. 19, 2014

(54) GELATIN-BASED MICROGELS

(71) Applicant: Agency for Science, Technology and Research, Singapore (SG)

(72) Inventors: Jun Li, Singapore (SG); Zhongxing Zhang, Singapore (SG); Chengde Liu, Singapore (SG)

(73) Assignee: Agency for Science, Technology and Research, Singapore (SG)

(21) Appl. No.: 14/103,421

(22) Filed: Dec. 11, 2013

(30) Foreign Application Priority Data
Dec. 14, 2012 (SG) 201209244-1

(51) Int. Cl. A61K 9/14; A61K 36/00; A61K 35/12

(52) U.S. Cl. CPC A61K 9/146; A61K 35/12; A61K 36/00
USPC 424/492; 424/93.7

(57) ABSTRACT

A gelatin-based microgel was prepared by a simple method starting from gelatin and alginate. The microgel was composed of a soft gelatin-based core coated by a permeable membrane. Gelatin-based core was moderately crosslinked by a novel crosslinker 4-arm PEG succinimidyl glutarate (pentaerytheritol core) (sPEG-4A-GS(10 k)).

### CLAIM OF PRIORITY

This application makes reference to and claims the benefit of priority of an application for “Gelatin-based microgels for cell encapsulation” filed on Dec. 14, 2012, with the Intellectual Property Office of Singapore, and its duly assigned application number 201209244-1. The content of said application filed on Dec. 14, 2012, is incorporated herein by reference for all purposes, including an incorporation of any element or part of the description, claims or drawings not contained herein.

### TECHNICAL FIELD

The present invention generally relates to methods for making microgels which have a use in cell delivery, tissue engineering or drug delivery. The present invention also relates to microgels having a biodegradable soft core, are coated by alternating permeable layers and which can be applied in the same field. The microgels are especially well-suited for cell encapsulation.

### BACKGROUND

A number of bioencapsulation technologies have been applied to protect tissues, cells, and biologically active compounds from potential hazardous processes in a physiological environment. After Chang proposed microencapsulation as an alternative to creating artificial tissues and organs in 1960s (Chang T M S. Semipermeable Microcapsules. Science. 1964; 146:524-525.), much attention has been attracted by the field of cells and bioactive compound immobilization.

In such systems, cells are encapsulated by a permeable shell that allows exchange of O₂, nutrients, and metabolites while it protects the inner cells from the host's immune system. It is important to provide an environment that mimics physiological conditions to maintain cellular functions. Cell delivery has been considered as the most promising approach while cells were cultured in matrix in 3-dimension environments. Microgels (from 100 μm to 500 μm) can be implanted in vivo (Huang S, Deng T Z, Wang Y J, Deng Z H, He L S, Liu S X, et al. Multifunctional implantable particles for skin tissue regeneration: Preparation, characterization, in vitro and in vivo studies. Acta Biomater. 2008; 4:1057-66. Gustafson C J, Birgisson A, Junker J, Huss F, Salemark L, Johnson H, et al. Employing human keratinocytes cultured on macroporous gelatin spheres to treat full thickness-wounds: An in vivo study on athymic rats. Burns. 2007; 33:726-35.). It could be beneficial in some applications for the long-term functionality of encapsulated cells. Furthermore, microgels can improve the permeability of oxygen and nutrients due to their large surface/volume ratio.

The simplest and most widely investigated approach is that alginate creates three dimensional structures when they react with multivalent ions. Alginates, naturally derived polysaccharides, are composed of (1-4)-linked β-D-mannuronic acid (M units) and α-L-guluronic acid (G units) monomers. The alginate molecule is a block copolymer composed of M-blocks, regions of G-blocks, and regions of a tactically organized M and G units. Cations, such as Ca²⁺, bind between the G blocks of adjacent alginate chains, creating ionic interchain bridges which induce gelation of aqueous alginate solutions.

Alginate is typically thought to be inert because they lack native ligands that could allow interaction with mammalian cells. Unfortunately, ionically crosslinked alginates lose mechanical stability over time in vitro, presumably due to an outward flux of crosslinking ions into the surrounding medium. In addition, physical crosslinking results in poor mechanical stability of hydrogel compared to chemical crosslinking. Therefore the use of alginates causes problems.

Gelatin is a commonly used polymer derived from collagen. It forms biocompatible and biodegradable hydrogels due to its biocompatibility and biodegradability with minimal immunological responses. Gelatin hydrogel was used to act as the extracellular matrix (ECM) to support the whole structure for cell encapsulation since gelatin which is inexpensive can promote cell adhesion and proteolytic degradation. Either UV-light or chemical crosslinker was used to create stable hydrogel. Photocrosslinked hydrogel may encounter a limitation in applications of deep tissue implants and its complex preparation process also limits its practical application. Many of crosslinking agents often elicit either cytotoxic side-effects or immunological responses from the host. For example, after implantation of the material, polymeric chains crosslinked by glutaraldehyde (GTA) are hydrolysed and monomeric GTA is released into the tissue, resulting in cytotoxicity and inflammation. This is another problem of the existing technical solutions for microgels.

There is a need to provide new microgels and methods for making them that overcome, or at least ameliorate, one or more of the disadvantages described above.

There is a need to provide biodegradable and biocompatible soft core gels that can be used in the above mentioned technical fields.

### SUMMARY

According to a first aspect, there is provided a method for preparing a microgel comprising the following steps:

(i) reacting a mixture comprising alginate and gelatin with an alginate physical crosslinker to form a crosslinked alginate microgel core

(ii) reacting the gelatin with a crosslinker to form a crosslinked gelatin microgel, and

(iii) removing the crosslinked alginate.

Advantageously, the gelatin microgels obtained with this method in which the initial alginate template is later removed show a better mechanical stability than the previously known alginate systems.

It has further been found that 4-arm PEG succinimidyl glutarate (pentaerytheritol core) can be used as chemical crosslinker for the gelatin microgel. It is non-cytotoxic and has soft segments in PEG backbone. The use of this crosslinker allows for a moderate crosslinking degree, appropriate gelling time, sufficient mechanical strength, and less solid stress induced by cell growth in the crosslinked microgel. These features are for instance beneficial to microgel fabrication and cell encapsulation.

Further advantageously, this method for making gelatin microgels is easier and more environmental friendly than the previously known water-in-oil emulsion technique, because it allows avoiding the use of organic solvents (oil) and surfactants during the preparation of the microgel.

According to a second aspect, a method has been found wherein step (i) is performed by air-driven droplet generation. Preferably, a mixture comprising alginate, gelatin and 4-arm PEG succinimidyl glutarate (pentaerytheritol core) is used in the droplet generation. More preferably the mixture may additionally contain cells or bioactive molecules.

Advantageously this new crosslinking way resulted in gelatin microgels with especially enhanced mechanical stability.

According to the third aspect, a method wherein the microgel core is additionally coated with alternate layers after step (ii) of the above mentioned process has been developed. Such alternate layer can be obtained preferably by coating with at least one polycation and at least one polyanion.

Advantageously, such permeable membrane coated microgel is a suitable cell carrier for cell encapsulation which allows for the cells to maintain over 80% of their viability for 9 days.

According to the fourth aspect, a microgel with a biodegradable and biocompatible core being coated by alternating layers has been obtained according to the invention.

Advantageously, the microgel shows a high mechanical stability and is suited for the encapsulation of cells, tissue and bioactive molecules.

### DEFINITIONS

The following words and terms used herein shall have the meaning indicated:

The term ‘microgel’ is to be interpreted broadly to include all types of gels with small particle size.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### Detailed Disclosure of Embodiments

Exemplary, non-limiting embodiments of a method for making the microgels and the microgels, will now be disclosed.

According to a first aspect, there is provided a method for preparing a microgel comprising the following steps:

(i) reacting a mixture comprising alginate and gelatin with an alginate physical crosslinker to form a crosslinked alginate microgel core

(ii) reacting the gelatin with a crosslinker to form a crosslinked gelatin microgel, and

(iii) removing the crosslinked alginate.

The term ‘microgel’ is to be interpreted broadly to include all types of gels with small particle size, preferably with a particle size of less than 1000 μm. More preferred less than 500 μm, and most preferred between 50 and 500 μm, with 80 to 120 μm particularly preferred. Gelatin preferable is Type A gelatin derived from porcine skin with gel strength about 300 g Bloom. Alginate preferable has the viscosity of 15-20 cP (concentration 1% in H₂O). [0028] For crosslinking alginates typical physical crosslinkers can be used. In one embodiment Ca²⁺ ions are preferably used as such crosslinker.

For crosslinking of gelatin all known gelatin crosslinker can be used, for instance in one such embodiment N-(3-dimethylaminopropyl)-N′-ethylcarbodiimide hydrochloride/N-hydroxysuccinimide (EDC/NHS), glutaraldehyde (GTA) or genipin.

According to one embodiment of the invention it has been found that 4-arm PEG succinimidyl glutarate (pentaerytheritol core) (sPEG-4A-GS) can be preferably used as the crosslinker for gelatin. 4-arm PEG succinimidyl glutarate (pentaerytheritol core) with an average molecular weight of about 10,000 Da (sPEG-4A-GS(10 k) is preferred. The crosslinking preferably takes place at a temperature of 30-50° C., more preferably about 37° C.

According to a second aspect, a method has been found wherein step (i) is performed by air-driven droplet generation. Preferably, a mixture comprising alginate, gelatin and 4-arm PEG succinimidyl glutarate (pentaerytheritol core) is used in the droplet generation.

Microgels with diameters of 50-500 μm are a preferred embodiment. Microgels with a diameter of about 100 μm are particular preferred. The inventive method is able to make such microgels with a diameter of about 100 μm with narrow size distribution.

In another embodiment the mixture for air-driven droplet generation may additionally contain cells or bioactive molecules. The cells are for instance mammalian or plant cells. The bioactive molecules are all molecules that show bioactivity and are preferably used in encapsulated form. Cells and/or biomolecules are encapsulated in the microgel core according to one inventive embodiment.

According to the third aspect, a method wherein the microgel core is additionally coated with alternate layers after step (ii) of the above mentioned process has been developed. Such alternate layer preferably is obtained by coating with at least one polycation and at least one polyanion. The resulting microgels preferably have diameters of 50-500 μm. Microgels with a diameter of about 100 μm are particularly preferred. The inventive method is able to produce such microgels with a diameter of about 100 μm with narrow size distribution.

Typical polycations include, but are not limited to natural polycations with chitosan or chitosan-containing materials being preferred.

Typical polyanions include, but are not limited to natural polyanions with alginate or alginate-containing materials being preferred.

Polycations, like chitosan, typically have an average molecular weight of 10 kDa, but are not limited to those. Polyanions, like alginate, typically have a viscosity of 15-20 cP (concentration 1% in H₂O, but are not limited to those.

Microgels obtained by a method according to any of the above mentioned aspects are also covered by the invention. According to the fourth aspect, a microgel with a biodegradable and biocompatible core being coated by alternating layers has been obtained according to the invention.

Said biodegradable and biocompatible core is according many of the embodiments of the invention of a soft type and preferably a chemically crosslinked gelatin. Typical crosslinkers are mentioned for the methods according to the invention above. The gelatin is preferably chemically crosslinked with 4-arm PEG succinimidyl glutarate (pentaerytheritol core). An average molecular weight of about 10,000 Da is preferred for the crosslinker.

The alternating layers can according to another embodiment be formed by coating the microgel with at least one polycation and at least one polyanion. Suitable polycations and polyanions are mentioned above for the methods according to the invention. 2 alternate layers are preferred. However, the invention is not limited to the chinotin/alginate type of alternate layer as described in the examples. Other alternate layers and/or additional layers can be used according to the invention.

The microgel core may comprise cells, tissue or bioactive molecules of the type described above for the methods according to the invention.

All microgels have a small particle size, according to preferred embodiments of less than 1000 μm. Microgels with diameters of 50-500 μm are preferred. Microgels with a diameter of about 100 μm are particular preferred. The inventive microgels with a diameter of about 100 μm with narrow size distribution are most preferred.

The microgels made according to the inventive methods and the microgels according to the invention can be used for drug delivery, tissue engineering or cell delivery due to their mechanical stability and physiological acceptance. A preferred embodiment is a cell delivery method wherein the cells are encapsulated in the microgel core.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1: Schematic illustration of preparation stages of gelatin-based microgels

FIG. 2. Optical images of (a) microgel-4(S2) and (b) microgel-4(S5). (scale bar: 100 μm)

FIG. 3. Zeta potential measurements for chitosan coating, alginate coating, and citrate treatment.

FIG. 4. Sizes of microgels at different preparation stages.

FIG. 5. Gel point measurement. Storage modulus and loss modulus of the crosslinked hydrogels with (a) sPEG-4A-GS(10 k) and (b) EDC/NHS as a function of time.

FIG. 6. Storage modulus and loss modulus of the crosslinked hydrogels with different crosslinkers (♦: G′ of hydrogel crosslinked by sPEG-4A-GS(10 k), ⋄: G″ of hydrogel crosslinked by sPEG-4A-GS(10 k), ▴: G′ of hydrogel crosslinked by EDC/NHS, ▴: G″ of hydrogel crosslinked by EDC/NHS).

FIG. 7. Swelling degrees (w/w) of microgels prepared in air-driven droplet generating method.

FIG. 8. Release of FITC-Dextrans with different molecular weights (40 and 70 kDa) from microgel-4(S5).

FIG. 9. Percentage of remaining microgels with and without chemical crosslinking as a function of vortexing time at 1800 rpm.

FIG. 10. Light micrographs of microgels containing 3T3 cells (0 day). Cells were encapsulated by microgel-4(S5). (Scale bar: 100 μm).

FIG. 11. Light and fluorescent micrographs of cell-containing microgel-4(S5) stained with LIVE/DEAD stains (2 days post-encapsulation). Live cells were stained green, and dead cells were stained red. (Scale bar: 100 μm)

FIG. 12. Cell viability measured using a LIVE/DEAD staining kit, as described herein.

### EXAMPLES

Non-limiting examples of the invention and a comparative example will be further described in greater detail by reference to specific Examples, which should not be construed as in any way limiting the scope of the invention.

### Examples

### Materials

Type A gelatin from porcine skin (˜300 g bloom), sodium alginate, Fluorescein isothiocyanate-dextrans (FITC-dextrans) (average molecular weight 4, 20, 40, and 70 kDa), N-(3-Dimethylaminopropyl)-N′-ethylcarbodiimide hydrochloride (EDC), N-Hydroxysuccinimide (NHS), sodium citrate dihydrate were obtained from Sigma-Aldrich Pte Ltd. sPEG-4A-GS(10 k) was purchased from JenKem Technology USA. The LIVE/DEAD reduced biohazard cell viability kit (Invitrogen L-7013) was ordered from Invitrogen. 4-arm polyethylene glycol (molecular weight 10 kDa) (sPEG-4A(10 k)) was supplied by Shearwater Polymers. Chitsoan (molecular weight 10 kDa) was provided by Haidebei Marline Bioengineering Co. Ltd., China. All other reagents were purchased from Sigma unless otherwise stated.

### Preparation and Characterization of Microgels According to the Invention

During the preparation of the gelatin-based microgels, an air-driven droplet generating method was adopted to gelatin cores according to the previously published method (Wikstrom J, Elomaa M, Syvajarvi H, Kuokkanen J, Yliperttula M, Honkakoski P, et al. Alginate-based microencapsulation of retinal pigment epithelial cell line for cell therapy. Biomaterials. 2008; 29:869-76). Microgels coated with chitosan and alginate were achieved according to the method described by Yu W T et al. (Yu W T, Song H Y, Zheng G S, Liu X D, Zhang Y, Ma X J. Study on membrane characteristics of alginate-chitosan microcapsule with cell growth. J Membrane Sci. 2011; 377:214-20). Mixed solution of sodium alginate (2.5%, w/v) and gelatin (3%, w/v) were prepared by suspending gelatin in sodium alginate solution containing NaCl (0.9%, w/v) at 37° C. The solution (0.2 mL) with or without crosslinker was dispersed through a 1 mL syringe and blunt ended cut needle (27 G, NIPRO, Osaka, Japan) connected to a nitrogen gas flow (300 L/h). The mixed solution was sprayed into 10 mL of CaCl₂ solution (100 mM) in a 15-mL glass vial. The microgels were washed with NaCl solution (0.9%, w/v) after 1 h in CaCl₂ solution. The microgels were immersed into 10 mL of chitosan solution (0.5%, w/v) for 10 min and washed with NaCl solution three times. The surface of microgel was further coated by incubating microgels in 10 mL of sodium alginate solution (0.125%, w/v) for 10 min. The microgels were washed with NaCl solution. After liquidized for 10 min using 55 mM sodium citrate and rinsed with NaCl solution, microgels were ready for further use. The assembly of chitosan and alginate layers at the surface of microgels was monitored by a Zetasizer (Nano Z S, Malvern Instruments, Malvern, UK). Zeta potential measurements were carried out in triplicate. Microgels were observed with optical phase-contrast microscope (Olympus 1×51) after preparation. At least 100 microgels were randomly selected for sizing of microgels using software ImageJ.

### Investigation of the Properties of Crosslinked Gelatin/Alginate Microgel

### Quantification of Free Amines Using Trinitrobenzenesulfonic Acid (TNBS) Assay

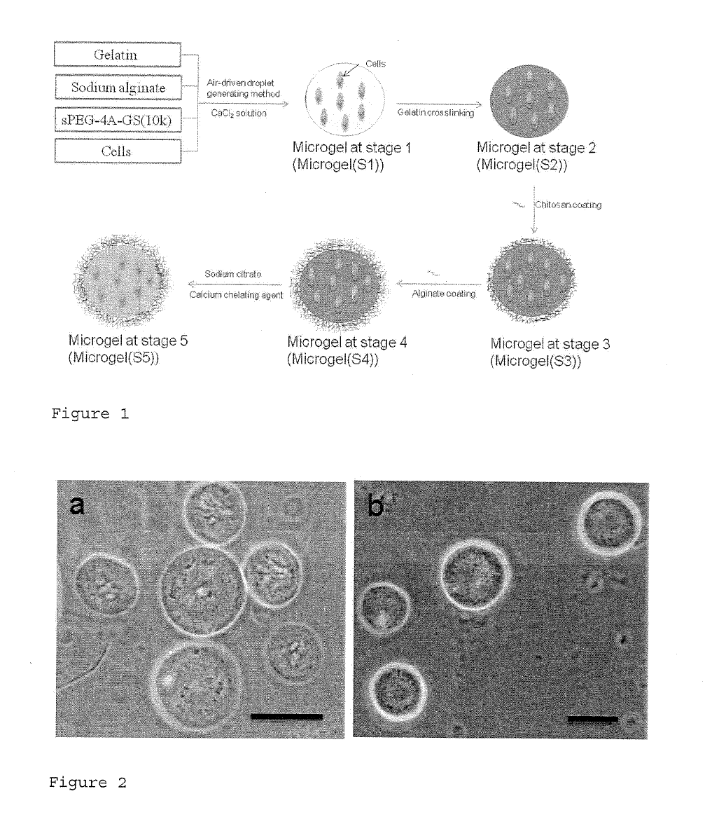

Gelatin and sodium alginate were dissolved in NaCl solution. sPEG-4A-GS(10 k) in NaCl solution was then added to the final concentrations of 0.003 mM. EDC/NHS crosslinker (0.006 mM/0.006 mM) was used as positive control. The solutions were incubated for 1 h at 37° C. to complete gelatin crosslinking reaction (FIG. 1). After that, the crosslinked hydrogel was further treated with 0.1 M glycine NaCl solution to block non-reacted aldehyde groups and washed three times with double-distilled water (Kang H W, Tabata Y, Ikada Y. Fabrication of porous gelatin scaffolds for tissue engineering. Biomaterials. 1999; 20:1339-44.). The measurement of crosslinking degree of gelatin hydrogel was determined by TNBS assay as previously described (Bubnis W A, Ofner C M. The Determination of Epsilon-Amino Groups in Soluble and Poorly Soluble Proteinaceous Materials by a Spectrophotometric Method Using Trinitrobenzenesulfonic Acid. Anal Biochem. 1992; 207:129-33.). Briefly, the hydrogel containing approximately 15 mg of gelatin was placed in a 15-mL screw cap test tube. 1 mL of NaHCO₃ (pH 8.5, 4%) and 1 mL of TNBS (0.5%) were added to the tubes and the mixtures were held at 40° C. for 4 h and 3 mL of 6 g/L HCl was added to change the pH value and the reaction mixture was autoclaved at 120° C. and 15-17 psi for 1 h. The trinitrophenyl (TNP)-ε-amino derivative is stable under these conditions (Kotaki A and Satake K. Acid and Alkaline Degradation of the TNP-Amino Acids and—Peptides. J. Biochem. 1964; 56: 299-306.). The obtained solution was then diluted with 5.0 mL of H₂O and extracted with 20 mL of diethyl ether three times to remove excess unreacted TNBS and TNP-α-amino groups. Ether was removed by heating the solution to 40° C. for 15 min in a water bath and the aliquot was diluted with 30 mL of H₂O and the absorbance was recorded at 346 nm in a Shimadzu UV-2450 spectrophotometer. The sample was read against reagent blank. The blank was prepared using the same method as the sample. In the blank preparation, HCl solution was added before the addition of TNBS solution to prohibit TNBS reacting with primary amino groups of gelatin. The crosslinking degree could be obtained from the differences between the absorbance values before and after crosslinking (Choi Y S, Hong S R, Lee Y M, Song K W, Park M H, Nam Y S. Study on gelatin-containing artificial skin: I. Preparation and characteristics of novel gelatin-alginate sponge. Biomaterials. 1999; 20:409-17.). The equation was the following:

$$Crosslinking degree ( \% ) = ( 1 - \frac{Absorbance of crosslinked hydrogel}{Absorbance of non \text{-} crosslinked hydrogel} ) \times 100$$

### Oscillatory Rheology Measurements

In order to identify the gelling time of the mirogel, rheological measurements were performed at 37° C. using a Rheostress RS600 (Haake Thermo Electron Corporation GmbH, Karlsruhe, Germany) with parallel plate geometry (20 mm diameter) at a gap of 1 to 2 mm as described before (Collin E C, Grad S, Zeugolis D I, Vinatier C S, Clouet J R, Guicheux J J, et al. An injectable vehicle for nucleus pulposus cell-based therapy. Biomaterials. 2011; 32:2862-70.). Briefly, the mixed solution was added to the plate at 37° C. after different crosslinkers were dissolved in alginate/gelatin mixed solution. The test methods employed were oscillatory time and stress sweep at 37° C. Time sweep experiments were carried out to determine the storage (G″) and loss (G″) moduli at 37° C. The measurements of the G″ and G″ moduli during the gelation were recorded as a function of time at a constant stress of 1 Pa for different crosslinkers. As soon as the mixed solution was introduced onto the plate, the data were collected at 4 min. The gel point was defined as the time when G″ was equal to G″ (Moura M J, Figueiredo M M, Gil M H. Rheological study of genipin crosslinked chitosan hydrogels. Biomacromolecules. 2007; 8:3823-9.). The impact of different crosslinkers on G″ and G″ was investigated. EDC/NHS crosslinker was used as a control. After the mixed solution containing crosslinker was loaded onto the rheometer at 37° C. for 1 h, stress sweep experiments were performed to provide G″ and G″ moduli to determine the linear viscoelastic region at a constant frequency of 1 Hz.

### Swelling of Microgels at Stage 2

The swelling degrees of microgels with or without chemical crosslinking were measured using a method earlier described (Holland T A, Tabata Y, Mikos A G. In vitro release of transforming growth factor-beta 1 from gelatin microparticles encapsulated in biodegradable, injectable oligo(poly(ethylene glycol) fumarate) hydrogels. Journal of Controlled Release. 2003; 91:299-313; Wei L, Cai C H, Lin J P, Wang L Q, Zhang X M. Degradation controllable biomaterials constructed from lysozyme-loaded Ca-alginate microparticle/chitosan composites. Polymer. 2011; 52:5139-48.). The microgels (lyophilized weight: around 5 mg) at stage 2 were collected by centrifuge and immersed in 0.5 mL of distilled water for 24 h. No obvious weight increase was observed after 24 h. Swollen microgels reached the swelling equilibrium. These swollen microgels were collected by centrifugation, gently wiped with filter paper to remove the water on the surface, and placed in a vial of known weight (Wv). The weight of swollen microgels and vial was recorded (Ws,v). The amount of water which was trapped in the gaps of microgels is very little and negligible. The weight of lyophilized microgels and vial was recorded (Wl,v). The swelling degree was then calculated as follows,

$$S = \frac{{W}_{s , v} - {W}_{v}}{{W}_{1 , v} - {W}_{v}}$$

### Microencapsulation of FITC-Dextran

FITC-dextrans (average molecular weights 4, 20, 40, and 70 kDa) were used to study the permeability of microgel-4 (Wikstrom J, Elomaa M, Syvajarvi H, Kuokkanen J, Yliperttula M, Honkakoski P, et al. Alginate-based microencapsulation of retinal pigment epithelial cell line for cell therapy. Biomaterials. 2008; 29:869-76.). Molecular weight had influence on the release rate of FITC-dextrans from the microgels.

Solutions of FITC-dextrans (0.25 mg/mL) in mixed solutions of sodium alginate (2.5%, w/v) and gelatin (3%, w/v) in NaCl solution were prepared by dissolving FITC-dextran in the mixed solution of sodium alginate and gelatin. After sPEG-4A-GS(10 k) was added into the mixed solution at a concentration of 0.003 M, 0.2 mL of the mixed solution was dispersed through a 1 mL syringe with a blunt ended cut needle. The preparation method was the same as the former preparation of microgels. To determine the encapsulation efficiency (EE), the microgel was dispersed in PBS buffer with 20 units/mL of collagenase and 0.36 mM of CaCl₂, and incubated at 37° C. for 1 h to degrade gelatin. The microgels were destructed completely overnight through the method in mechanical stability test. The residues were isolated by centrifuging the sample at 1000 rpm for 10 min. The concentration of FITC-dextran in the supernatant was determined by measuring fluorescence of the samples at the excitation and emission wavelengths of 485 and 520 nm, respectively, with a BMG FLUOstar Optima multimode plate reader with accompanying BMG FLUOstar Optima Version 1.20 software (BMG Labtechnologies, Durham, N.C.).

The release experiments were carried out at 37° C. in water bath at the rotation speed of 140 rpm. Microgels were dispersed in 1 mL of PBS buffer. The release of the FITC-dextran was determined by periodically refreshing 0.1 mL of release medium. The released FITC-Dextran was determined using a BMG FLUOstar Optima multimode plate reader by the above measuring method.

### Mechanical Stability Test of Microgels

The mechanical stability of microgels was evaluated by agitation of 0.5 mg of lyophilized microgels in a plastic vial together with 0.7 mL of PBS on a vortex mixer at about 1800 rpm. Two polystyrene particles (diameter: 0.5 mm) were added to increase the force to break the microgels. At various time intervals, the vial was removed from the mixer and 0.1 mL was extracted for counting the number of remaining microgels under phase-contrast microscope. The vial was placed back on the vortex mixer and continued for agitation. Three independent experiments were performed for each microgel.

### Encapsulation of 3T3 Cells, Cell Culture, and Cell Viability Assay

Cell pellet was suspended in the mixed solution of sodium alginate (2.5%, w/v) and gelatin (3%, w/v). After the crosslinker was added, the final cell density was 2×10⁵ cells/mL. Cell encapsulation was conducted according to the microgel preparation method described above. The encapsulated 3T3 cells were cultivated in DMEM containing 10% fetal bovine serum (FBS) and 1% penicillin/streptomycin (PS) for 12 days. Viability of the encapsulated 3T3 cells was assessed by LIVE/DEAD kit (Invitrogen L-7013). Briefly, non-viable cells were stained red and viable cells were stained green at predetermined time points. Cell viability was assessed by counting the number of green cells over the total number of cells and expressed as a percentage (n=20 microgels). Samples were analyzed under a fluorescence microscope (Olympus IX71, Olympus Optical Co. Ltd, Tokyo, Japan.).

### Results

### Characterization of Microgels

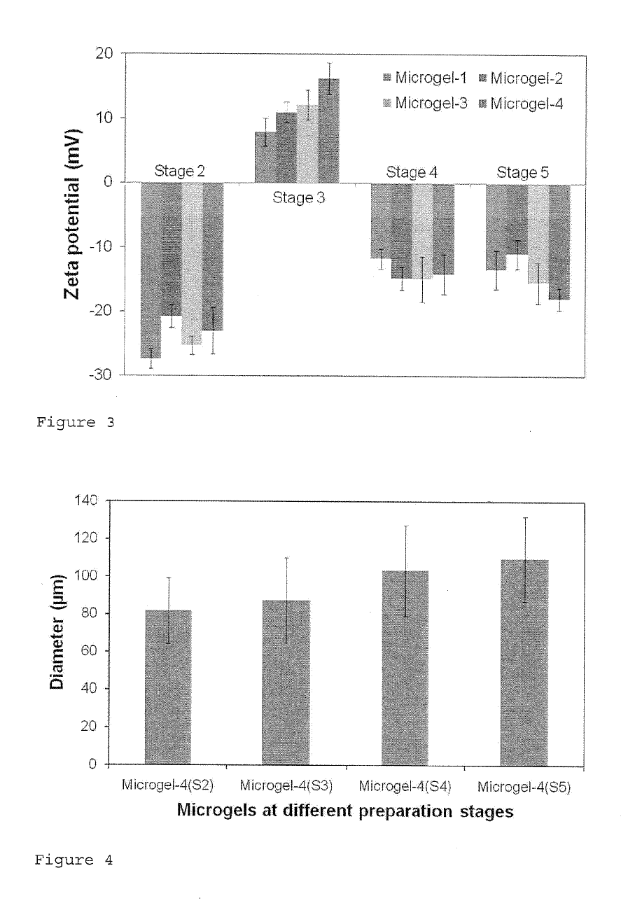

The formulations of microgels were tabulated in Table 1. Sodium alginate solution or mixed solution of sodium alginate and gelatin containing crosslinker or sPEG-4A(10 k) was sprayed into CaCl₂ solution to produce microgels at Stage 1. After held for 1 h at 37° C., the microgels at Stage 2 were recovered by centrifuge and rinsed in NaCl solution. The microgels were found to be stable in NaCl solution after microgel recovery (FIG. 2), and exhibited negative surface charge (FIG. 3). So the microgel surface could be used for polyelectrolyte Layer-by-Layer self-assembly to generate protective membrane for use in biomedical application.

TABLE 1
Size measurement of microgels

| | | Microgel | Microgel |
|---|---|---|---|
| Micro- | | (S2) | (S5) |
| gels | Formulations | size (μm) | size (μm) |
| Microgel-1 | Sodium alginate (2.5%, w/v) | 55 | 86.6 |
| Microgel-2 | Sodium alginate (2.5%, w/v) + | 98.5 | 123.5 |
| | Gelatin (3%, w/v) + EDC | | |
| | (0.006M)/NHS (0.006M) | | |
| Microgel-3 | Sodium alginate (2.5%, w/v) + | 87.7 | 109.4 |
| | Gelatin (3%, w/v) + sPEG-4A | | |
| | (10k) (0.003M) | | |
| Microgel-4 | Sodium alginate (2.5%, w/v) + | 81.7 | 103.3 |
| | Gelatin (3%, w/v) + sPEG-4A-GS | | |
| | (10k) (0.003M) | | |

The microgels at stage 2 were immersed in chitosan solution (molecular weight about 10,000 Da) and rinsed three times in NaCl solution to produce the microgels at stage 3. Alginate coating was then added by incubating the microgels at stage 3 in sodium alginate solution to bind positively charged chitosan on the microgel surface and the microgels at stage 4 were washed with NaCl solution. The microgels at stage 5 were formed after citrate treatment. Phase-contrast microscope was used to image the microgels. The representative microscopic images of microgels were shown in FIG. 2. It can be observed that spherical microgels were intact with smooth surface. At least 100 microgels were randomly selected for size measurement using software ImageJ, which provided diameters of microgels. It was found that the diameters of the microgels were relative uniform around 100 μm (Table 1). FIG. 4 shows the diameters of microgels at different preparation stages of microgel-4.

The self-assembly of chitosan and alginate layers was monitored by Zetasizer (Nano Z S, Malvern). Chitosan and alginate were deposited on the surface of microgels, and the zeta potentials of the microgels were observed to change from about −30 mV before chitosan coating to about +15 mV after chitosan coating (FIG. 3), which indicates the formation of the desired layer. This result indicates that the microgel cores had enough negative surface charge after gelation. The surface charge made chitosan and alginate deposition onto microgels straightforward. The polyelectrolyte layer may provide a protective membrane for encapsulated biomolecules and cells and stabilize microgels against dissolution in a biological environment.

### Measurement of Crosslinking Degree

sPEG-4A-GS(10 k) molecule is a pegylated structure presenting four terminal N-hydroxysuccinimidyl (NHS) reactive groups. NHS terminal groups react with the amine groups of gelatin inducing crosslinking (Scheme 1). TNBS reacts with primary amines of gelatin rather than the hydroxyl groups or the imidazole nitrogens and the resulting TNP-S-derivative is labile while TNBS reacted with sulfhydryl groups. TNP-α-amino groups can be removed from the aqueous phase through the ether extraction. The specific determination of ε-amino groups was allowed after the removal of TNP-α-amino groups. The duration of the TNBS reaction time was required for the completion of the TNBS reaction with crosslinked and poorly soluble gelatin. Water-soluble sPEG-4A-GS(10 k) and EDC/NHS crosslinker have been used as crosslinking agents in biomedical application (Damink LHHO, Dijkstra P J, vanLuyn M J A, vanWachem P B, Nieuwenhuis P, Feijen J. In vitro degradation of dermal sheep collagen crosslinked using a water-soluble carbodiimide. Biomaterials. 1996; 17:679-84.). Considering the crosslinking mechanism by EDC/NHS, alginate could be also crosslinked since carboxyl groups rich in alginate could attack EDC. The crosslinking efficiency is not affected when the crosslinker sPEG-4A-GS(10 k) was applied. The degrees of crosslinking were tabulated in Table 2. The crosslinking efficiency of EDC/NHS was higher than that of sPEG-4A-GS(10 k), while the molar ratio of sPEG-4A-GS(10 k) to EDC was 1:2. It may be due to the fact that some of functional groups of sPEG-4A-GS(10 k) were less active in the reaction.

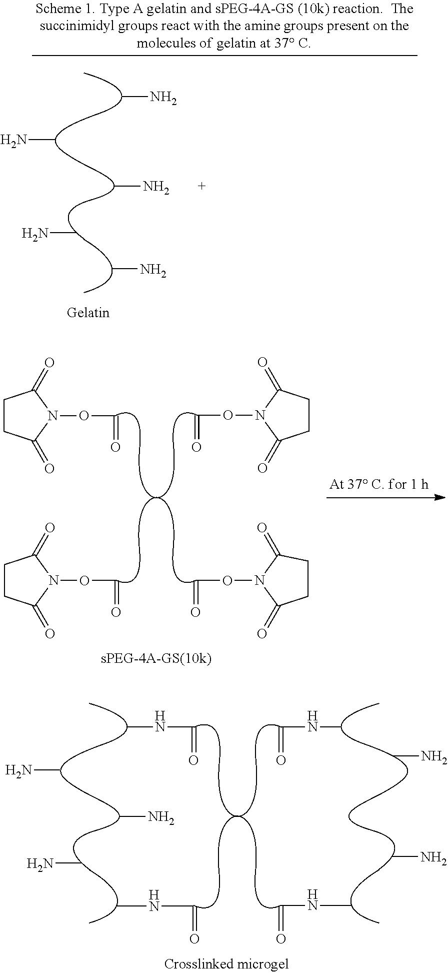

TABLE 2
Gelling time and crosslinking degree

| Samples | Crosslinking degree | Gelling time (min) |
|---|---|---|
| Hydrogel crosslinked by | 31.4% | 36 |
| sPEG-4A-GS (10k) | | |
| Hydrogel crosslinked by | 45.6% | 4.9 |
| EDC/NHS | | |

### Rheological Analysis of Gelatin/Alginate Microgel

The measurements of rheological properties are very useful to characterize the sol-gel transition (Montembault A, Viton C, Domard A. Rheometric study of the gelation of chitosan in a hydroalcoholic medium. Biomaterials. 2005; 26:1633-43; Liu K L, Zhu J L, Li J. Elucidating rheological property enhancements in supramolecular hydrogels of short poly[(R,S)-3-hydroxybutyrate]-based amphiphilic triblock copolymer and alpha-cyclodextrin for injectable hydrogel applications. Soft Matter. 2010; 6:2300-11; Esfahani H B, Yekta B E, Marghussian V K. Rheology and gelation behavior of gel-cast cordierite-based glass suspensions. Ceram Int. 2012; 38:1175-9.). Dynamic rheometry is a tool which allows properties to be probed without disruption of the microstructure (Moura M J, Figueiredo M M, Gil M H. Rheological study of genipin crosslinked chitosan hydrogels. Biomacromolecules. 2007; 8:3823-9.). It is also a method for studying the viscoelastic material functions, such as dynamic shear moduli G″ and G″. The two viscoelastic material functions were defined as follows:

G′=σ₀ cos(δ)/γ₀

G″=σ₀ sin(δ)/γ₀

Where σ₀ is the stress, γ₀ is the strain amplitude and δ is the phase angle between stress and strain. G′ is storage modulus which measures the elastic energy representing the elastic portion and G″ is loss modulus which measures the dissipated energy representing the viscous portion (Ramazani-Harandi M J, Zohuriaan-Mehr M J, Yousefi A A, Ershad-Langroudi A, Kabiri K. Rheological determination of the swollen gel strength of superabsorbent polymer hydrogels. Polym Test. 2006; 25:470-4.). The relative magnitudes of G″ and G″ will provide information regarding the proportion of the stored energy and dissipated energy during the flow over each cycle of frequency oscillation that indicates the overall viscoelasticity of the sample.

Attention has been focused on the gel point determination in polymer crosslinking systems. Sol-gel transition of polymer gels can be monitored from the development of the viscoelastic functions of the material at the gel point which can be characterized by the appearance of one macromolecule chain with infinite molecular weight in an reactive system (Zhao Y, Cao Y, Yang Y L, Wu C. Rheological study of the sol-gel transition of hybrid gels. Macromolecules. 2003; 36:855-9.). It can be determined by the viscoelastic properties change abruptly from a liquid-like state to a solid-like state (Matricardi P, Dentini M, Crescenzi V. Rheological Gel-Point Determination for a Polysaccharide System Undergoing Chemical Crosslinking. Macromolecules. 1993; 26:4386-7.). The gel point coincides with the point at which G′ equals to G″.

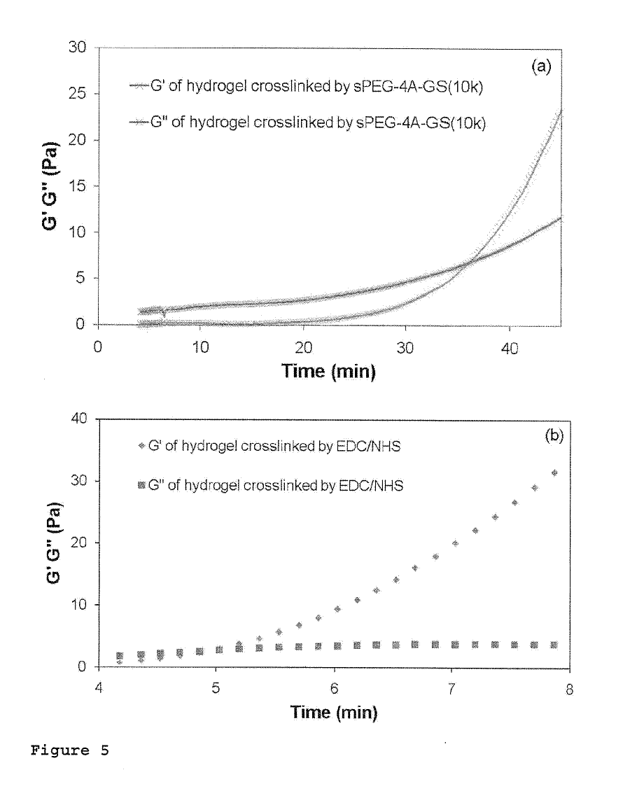

To improve knowledge regarding the gelation kinetics and properties of microgels in reactive system, the rheology properties of gelatin/alginate hydrogels were studied. Storage modulus G′ and loss modulus G″ were monitored. FIG. 5 shows the time sweep profiles of G′ and G″ moduli for alginate/gelatin solution versus crosslinking time. The rheological test was started 4 min after the crosslinker was added to the mixed solution of gelatin and alginate. At the beginning, G′ was larger than G″, which indicated the sample was still in a liquid state and viscous properties dominated. The liquid-like state began to turn into a gel-like state because of the formation of the crosslinked network near the gel point. The elastic properties began to dominate the viscous properties after a G′ and G″ crossover. The time required for G″ and G″ to increase to the crossover was the gelling time. From FIG. 5a, it was obviously observed that EDC/NHS crosslinker led to gelling time of 4.9 min while the gelling time of hydrogel crosslinked by sPEG-4A-GS(10 k) was 36 min, indicating high reactive activity of EDC/NHS crosslinker. Experiments were carried out in triplicate. Gelling time of the precursors plays a key role in the design of our microgels. Short gelling time can lead to big and non-circular microgels with the droplet generation method. Thus, sPEG-4A-GS(10 k) can be used in optimally designing the microgel with respect to the gelling time.

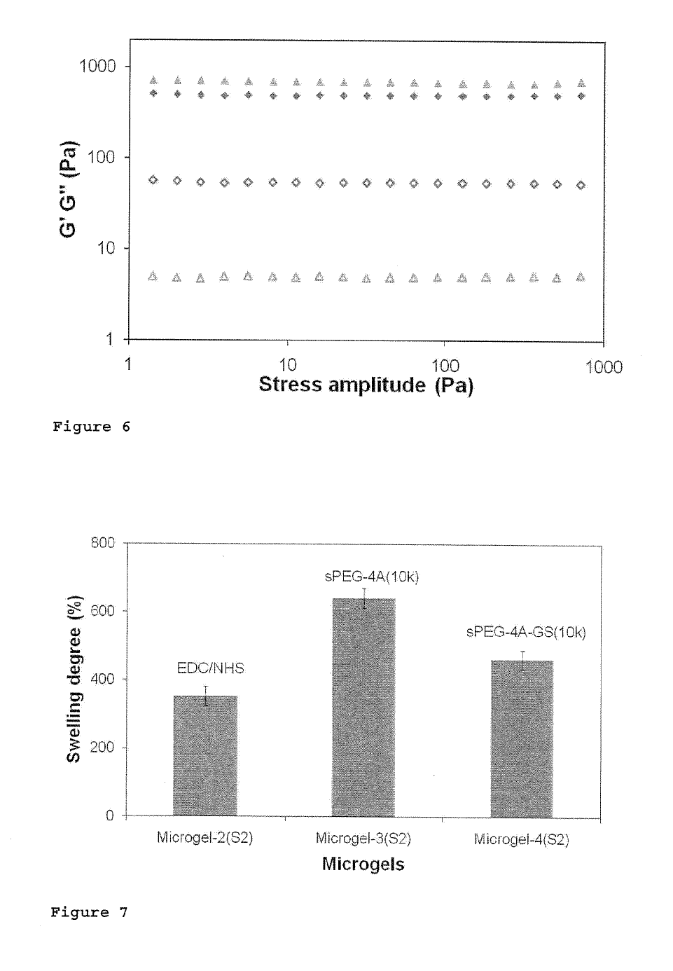

The stress range over which G′ and G″ are independent of the applied shear stress is the linear viscoelastic region (Moura M J, Figueiredo M M, Gil M H. Rheological study of genipin crosslinked chitosan hydrogels. Biomacromolecules. 2007; 8:3823-9.). The linear viscoelastic region profiles of crosslinked hydrogel were shown in FIG. 6. G′ and G″ were quite independent of stress in the linear viscoelastic region. G′ values were always larger than G″ values over the shear stress range from 1 to 1000 Pa, which confirmed their gel states. As shown in FIG. 6, an oscillatory stress sweep measurement shows that the hydrogel crosslinked by EDC/NHS within its linear viscoelastic range up to almost 1000 Pa in terms of shear stress had G′ dominating over G′ of the hydrogel crosslinked by sPEG-4A-GS(10 k). G′ represents the elastic portion in viscoelastic hydrogel which is correlated with the crosslinking degree in the hydrogel network. The high G′ values of the hydrogel were due to its high crosslinking degree (Table 2). The hydrogel crosslinked by EDC/NHS had high G′ values, which indicates it was much stiffer (Liu K L, Zhu J L, Li J. Elucidating rheological property enhancements in supramolecular hydrogels of short poly[(R,S)-3-hydroxybutyrate]-based amphiphilic triblock copolymer and alpha-cyclodextrin for injectable hydrogel applications. Soft Matter. 2010; 6:2300-11.). Observed from FIG. 6, the hydrogel crosslinked by EDC/NHS had the lower ratio of G″ to G′ than the hydrogel crosslinked by sPEG-4A-GS(10 k), which means that the former is more solid-like than the latter, indicating predominant elastic behavior. The more solid-like property can be attributed to the higher crosslinking degree in the hydrogel crosslinked by EDC/NHS, as proven by TNBS assay. In addition, incorporating ‘zero length’ amide crosslinks can also make the hydrogel stiffer than the incorporation of sPEG-4A-GS(10 k) crosslinker with soft PEG segment.

The stress generated by cellular growth in the hydrogel with more solid-like property may cause some biological limitations including long-term stability and reduced cell growth in the application of cell encapsulation. Thus, it is expected that hydrogel crosslinked by sPEG-4A-GS(10 k) is more suitable for cell encapsulation.

### Swelling Properties of Microgels

Microgel swelling experiments revealed a significant difference in swelling degrees of different microgels. Swelling degree is a reflection of the crosslinking degree in hydrogel system. In principle, the polymer gel becomes more compact when the crosslinking degree increases, leading to a decrease of microgel swelling degree. Crosslinking resulted in low swelling degree of gelatin-alginate microgels. In the preparation of the microgels, different crosslinkers are influencing the properties of microgels. We investigated the dependence of swelling degrees on different crosslinkers. FIG. 7 presents the swelling degree of the microgels with or without chemical crosslinking. As shown in FIG. 7, the swelling degree of microgel-4(S2) crosslinked by sPEG-4A-GS(10 k) was higher than it of microgel-2(S2) crosslinked by EDC/NHS crosslinker. In hydrogel system, swelling degree reflects the crosslinking degree of hydrogel. In principle, high crosslinking degree results in more compact polymer gel networks. The change of swelling degree caused by the crosslinking degree can affect cell viability through changing water content of a hydrogel. Hydrogel porosity can be reflected by the water content of a hydrogel. High porosity of microgel is beneficial to cell culture. The expected result is that an acceptable environment containing high water content could sustain cell viability inside the hydrogel matrix. The swelling degree of microgel-3(S2) was significantly greater than the other two crosslinked microgels. However, the low mechanical stability of uncrosslinked microgel limits its application in cell encapsulation.

### Permeability of Outer Membranes of Microgel

FITC-dextrans with different molecular weights were encapsulated and released to study the permeability of the outer membranes of gelatin-based microgels. Molecular weight had influence on the encapsulation and release of FITC-dextrans from the microgels. In FITC-dextran encapsulation, FITC dextrans with low molecular weights were not encapsulated in microgels, which may be due to the smaller sizes of the molecules compared to the pore size of the microgels. FITC-dextrans (average molecular weight 40 and 70 kDa) were encapsulated in the microgels successfully. Encapsulation efficiency was increased with enhancing the molecular weight of FITC-dextran (Table 3).

TABLE 3
Encapsulation efficiency of microgel-4(S5)

| | Molecular weight of FITC-Dextran (kDa) | | | |
|---|---|---|---|---|
| | 4 | 20 | 40 | 70 |
| Encapsulation efficiency (%) | 0 | 0 | 2.93 ± 0.81 | 7.49 ± 0.72 |

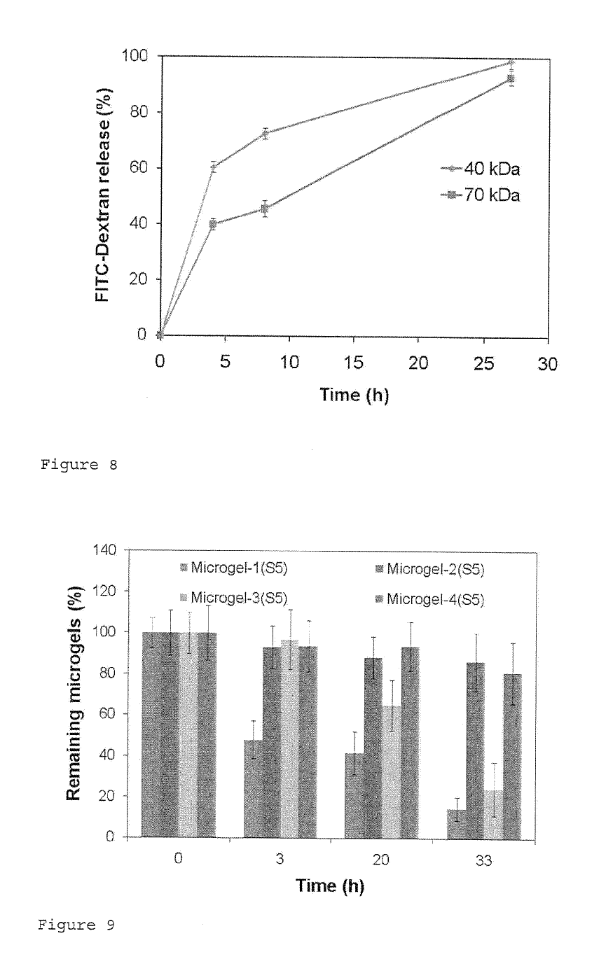

Most of FITC-dextrans of 40 and 70 kDa were released from microgel-4(S5) within 27 h (FIG. 8). The release was fast in the initial phase. After that, the release slowed down. The release rate of FITC-dextran (average molecular weight 40 kDa) was higher than that of FITC-dextran (average molecular weight 70 kDa). FITC-dextran with high molecular weight led to the slow release of FITC-dextran, which has been proved in a previous study. The pore size in the microgels allowed the diffusion of FITC-dextran of 70 kDa because 93% of it was released within 27 h. Thus, the microgel-4(S5) has the potential for cell delivery due to the permeability of the outer membranes of microgel.

### Mechanical Stability of Microgels

The mechanical stability of the microgels was evaluated by agitation of microgels on a vortex mixer. FIG. 9 shows the relative numbers of remaining microgels as a function of time. At 33 h, the remaining microgel-2(S5) and microgel-4(S5) were around 80% while the other remaining microgels were around 20%. At different intervals, more microgels without chemical crosslinking were fractured compared to chemically crosslinked microgels. It was observed that chemically crosslinked microgels were more difficult to deform. The microgel-3(S5) had the higher mechanical strength than microgel-1(S5), which may be due to unreleased gelatin and PEG enhancing its mechanical stability. The results indicate that the chemical crosslinking was efficient to strengthen the cores of the microgels. Thus, crosslinked microgels would be of great potential for cell delivery.

Encapsulation of Cells within Microgels

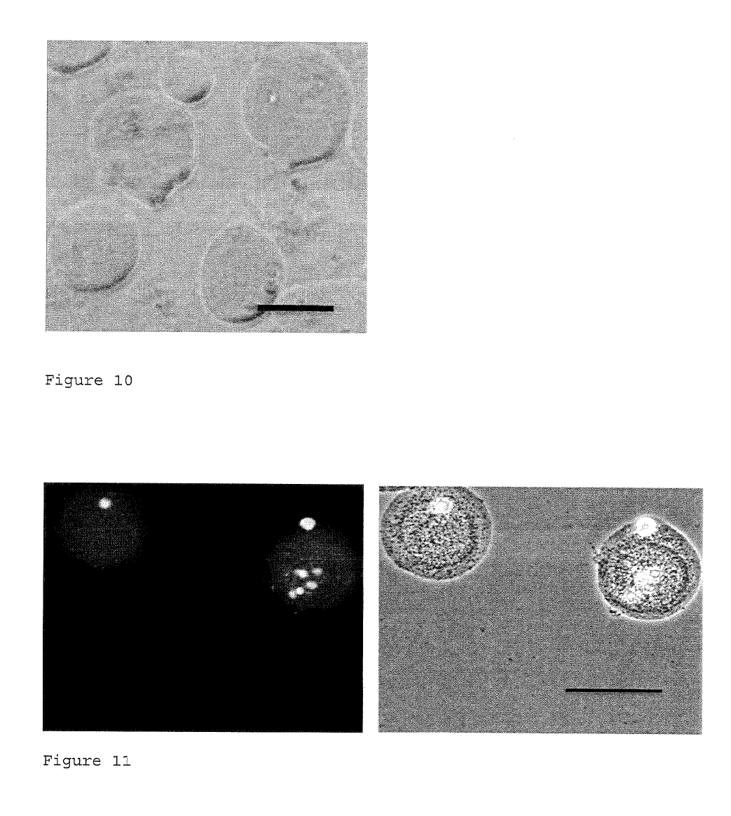

To validate the application of the cell-encapsulated microgels in the biomedical applications, 3T3 fibroblast cells were encapsulated within the microgels and the viability of cells was assessed by using a viability assay. Microgels for cell encapsulation were generated based on the conditions obtained from ‘Preparation of Microgels’ section. 3T3 cells were collected after trypsinization and resuspended in the mixed solution at a concentration of 2×10⁵ cells/mL. The solution containing 3T3 cells was dropped into calcium chloride solution to produce microgels. The microgels were found to be stable in NaCl solution after the removal of CaCl₂ solution and exhibited negative surface charge. So the microgels could be used as the microgel cores for polyelectrolyte to form protective membranes on the cores. All microgels were coated with chitosan and alginate. After citrate treatment, the microgels encapsulating cells were formed. FIG. 10 shows the representative microscopic image of Microgel-4(S5) encapsulating 3T3 cells.

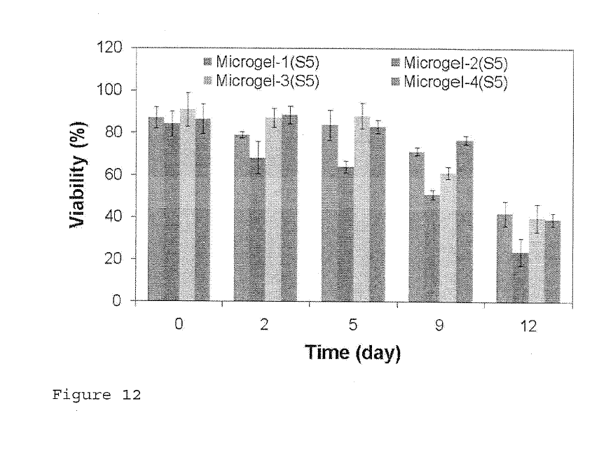

Cell viability was characterized by LIVE/DEAD kit (Molecular Probes, Invitrogen L-7013). Microgels encapsulating cells were collected at predetermined intervals. The encapsulated cells were stained with LIVE/DEAD kit (FIG. 11) and analyzed for cell viability. To learn about the influence of microgel fabrication process on the cell viability, cell viability at stage 5 was checked. The viability of the cells was not qualitatively different after preparation stages. It is observed that cell viability was remained above 80% (FIG. 12). The result indicates cell-friendly feature of the cell encapsulation method. The preparation stages did not result in a significant amount of cell death, similar to previously published studies (Costa N L, Sher P, Mano J F. Liquefied Capsules Coated with Multilayered Polyelectrolyte Films for Cell Immobilization. Adv Eng Mater. 2011; 13:B218-B224.).

Most of the cells were encapsulated in the core of the microgel. In the long-term (>2 days) cell viability tests, the 3T3 cells in microgel-4(S5) were viable for at least 9 days in in vitro culture. 3T3 cells cultured in microgel-1(S5) for 12 days served as the control. Cell culture in the microgels like microgel-1(S5) is accepted as a method for successfully encapsulating Human osteoblast-like cells, Human embryonic stem cells and so on. Clumps of cells, as well as individual cells, were observed in microgel-4 (S5) similar to microgel-1(S5). Some of the microgel-1(55) were broken at 5 day and cells were released, which is due to the low mechanical stability of liquid-core microgel-1(S5). At 12nd day, most of released cells from microgel-1(55) adhered to the bottom of 24-well culture plates and proliferated. Chemical crosslinkers, such as EDC/NHS and GTA, were commonly used for stabilization. However, they were not allowed in injectable hydrogel. From the cytotoxicity result, no cytotoxicity of sPEG-4A-GS(10 k) was observed with cell viability over 80% compared to the crosslinker EDC/NHS at 2 day. The crosslinker sPEG-4A-GS(10 k) is more suitable than GTA in the application of injectable hydrogel. sPEG-4A(10 k) was used as a control to study the influence of crosslinking on cell viability. There is no significant difference between microgel-3(S5) and microgel-4(S5). Therefore, the gelatin-based microgel would be a suitable cell carrier for cell delivery.

### Applications

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

What is claimed is:

1. A method for preparing a microgel comprising the following steps:
(i) reacting a mixture comprising alginate and gelatin with an alginate physical crosslinker to form a crosslinked alginate microgel core;
(ii) reacting the gelatin with crosslinker to form a crosslinked gelatin microgel; and
(iii) removing the crosslinked alginate.

2. The method according to claim 1 wherein the crosslinker in step (ii) is a 4-arm PEG succinimidyl glutarate (pentaerytheritol core).

3. The method according to claim 1 wherein step (i) is performed by air-driven droplet generation.

4. The method according to claim 3 wherein a mixture comprising alginate, gelatin and 4-arm PEG succinimidyl glutarate (pentaerytheritol core) is used in the droplet generation.

5. The method according to claim 4 wherein the mixture additionally contains cells or bioactive molecules.

6. The method according to claim 1 wherein the microgel core is additionally coated with alternate layers.

7. A microgel obtained by a method according to claim 1.

8. A microgel with a biodegradable and biocompatible core being coated by alternating layers.

9. The microgel according to claim 8, wherein said biodegradable and biocompatible core is a chemically crosslinked gelatin.

10. The microgel according to claim 9, wherein the gelatin was chemically crosslinked with 4-arm PEG succinimidyl glutarate (pentaerytheritol core).

11. The microgel according to claim 8, wherein the alternating layers are composed of at least one polycation and at least one polyanion.

12. The microgel according to claim 11, wherein the polycation is chitosan or chitosan-containing material, and the polyanion is alginate or alginate-containing material.

13. The microgel according to claim 8, wherein the number of alternating layers is at least 2.

14. The microgel according to claim 8, additionally comprising cells, tissue or bioactive molecules in the microgel core.

15. The microgel according to claim 8, in the form of particles of a size of 50 to 500 μm.

16. A method of drug delivery, tissue engineering or cell delivery wherein a microgel according to claim 7 is used.

17. A method of drug delivery, tissue engineering or cell delivery wherein a microgel according to claim 8 is used.

18. A cell delivery method according to claim 16 wherein the cells are encapsulated in the microgel core.

19. A cell delivery method according to claim 17 wherein the cells are encapsulated in the microgel core.
